(19) United States
(12) Patent Application Publication
(10) Pub. No.: US 2022/0323311 A1
(43) Pub. Date: Oct. 13, 2022

(54) PERSONAL HYGIENE WIPE WITH INTEGRAL URINE GLUCOSE DETECTION FEATURE

(71) Applicant: Georgia Crawford, Edgewater (JM)

(72) Inventor: Georgia Crawford, Edgewater (JM)

(21) Appl. No.: 17/472,971

(22) Filed: Sep. 13, 2021

Related U.S. Application Data
(60) Provisional application No. 63/172,784, filed on Apr. 9, 2021.

(51) Int. Cl. A61K 8/02; A61Q 17/00; C12Q 1/26; C12Q 1/30; A61Q 19/10

(52) U.S. Cl. CPC A61K 8/0208; A61Q 17/005; C12Q 1/26; C12Q 1/30; A61Q 19/10

(57) ABSTRACT

A personal hygiene wipe with an integral urine glucose detection feature. The wipe includes a substrate that is adapted for impregnation with a composition having one or more drying, cleaning, odor control, or antibacterial properties. The wipe also is saturated with a chemical composition for detecting glucose in urine. When glucose is detected, the wipe turns to a different color.

### CROSS-REFERENCE TO RELATED APPLICATION

This application claims priority to provisional application Ser. No. 63/172,784 filed on Apr. 9, 2021 and entitled “SaveALife—Diabetic Toilet Paper. SaveALife: Diabetic Toilet Paper/Wipe is a toilet paper for female diabetics, used after urinating which will change color if the user has abnormal glucose levels in their urine. It is a rapid glucose test in toilet paper form”. Said application is incorporated herein by reference.

### TECHNICAL FIELD

The present invention generally relates to the fields of bathroom hygiene and self-administered medical screening. More particularly, the invention is directed to a personal hygiene wipe with an integral urine glucose detection feature.

### BACKGROUND OF THE INVENTION

The need for bathroom hygiene is as old as mankind and throughout history that need has been satisfied in accordance with local custom, social hierarchy and the availability of natural tools and material. The early days of wiping with stones, seashells and animal fur progressed through leaves and plants to modern-day specially engineered toilet paper, and now toilet wipes.

While men and women typically use toilet paper, or a suitable wipe, after a bowel movement, many women also use toilet paper after urinating as part of their normal hygiene regiment. In additional to hygiene, wiping the vaginal area after urination is an important step in the prevention of urinary tract infections for women. Proper bathroom hygiene is especially important for women, given the female urethral opening's close proximity to the rectum and the absence of a female appendage to aim the flow of urine away from the body as is the case with males. If the vaginal area is not cleaned after urination, urine droplets will accumulate in the pubic region and be transferred to undergarments. In addition to causing odor, the accumulation of urine creates a breeding ground for bacteria which increases the risk of a urinary tract infection in females.

It is also noted that it is nearly impossible for a human to have a bowel movement without also passing some amount of urine. One can urine without having a bowel movement, but usually not the other way around. This has to do with how the larger circular muscles around the anus involuntarily flex and interact with the much smaller circular muscles around the urethra during a bowel movement in both men and women. Thus, every bowel movement will require toilet paper or a toilet wipe to clean the anal area and in the case of women, the vaginal area to dry any residual urine.

Research and numerous surveys show that proper bathroom hygiene is not always observed by most women and not at all by some. While there is better adherence to proper hygiene following a bowel movement, adherence drops off when voiding only urine. Thus, many women do not dry their vaginal area after urination and rely on their underwear to catch the residue. Doing so, however, is a bad practice as it increases the risk of a urinary tract infection as explained above.

Toilet paper is readily available virtually everywhere, biodegrades quickly and efficiently, and does not damage pipelines, sewage systems, and water treatment centers.

While traditional toilet paper has its advantages, it has disadvantages as well. A noted disadvantage is that in order to achieve a thorough cleaning, many users use more paper than necessary. In addition, the white color of toilet paper is often the result of it being bleached with chlorine which can produce a harmful chemical by-product known as dioxin.

Thus, wipes are increasingly being used as an alternative to toilet paper. As there currently is no recognized standard, wipes come in a variety of sizes, shape and constructions depending on brand and marketing strategy.

Wipes have the advantage of being sturdier than toilet paper and their moistness helps in the cleaning process which results in a greater sense of feeling clean. However, poorly designed flushable wipes create problems for sewer pipes and water treatment systems and for the environment as well.

Wet wipes are said to be a major factor in the creation of fatbergs. Fatbergs are larges masses of non-biodegradable solid waste in a sewage system. They are formed of congealed fat and personal hygiene products that have been flushed down toilets.

With respect to the environment, plastic is a common material used to make many flushable wipes. When the wipes break down after being flushed down the toilet or otherwise discarded, they release microplastics into the water or ground which find their way into the food system and ultimately into the human body. In addition, there is the adverse impact of plastic pollution on marine life.

As mentioned above, the present invention also relates to the field of self-administered medical screening.

Health care has many stakeholders, including patients, healthcare provides, insurance companies and of course governments. In the United States, the majority of health care services are delivered through private entities as the U.S. does not have a universal health care system. In many cases, governments ultimately bear a great deal of the cost of health care. Through its regulatory powers, governments also control many aspects of health care accessibility and delivery.

While every stakeholder strives for the best healthcare possible for its constitutes, each stakeholder also has its own particular interest to protect, often at the expense of other stakeholders.

Thus, the natural tension between health care stakeholders often leaves many of the weakest stakeholders, i.e., patients, without adequate healthcare care or no health care at all. Thus, more of the health care burden is increasingly being placed on the end user of health care services.

For example, vigilance of health care needs is increasingly being placed on individuals through better diet, more physical exercise and more attention to the signs and signals that the human body provides that indicate a potential health care problem. For example, high blood pressure is the body's way of signaling that one's cardiovascular system is in need of attention. A rapid heart rate that is unrelated to physical exercise and an irregular heart beat are others. These conditions are all detectable in the first instance by a person using readily available over the counter diagnostic devices and do not require a lab or doctor's visit. However, a doctor's visit is often required, or at least recommended, to confirm the condition and provide subsequent treatment.

Other signs of a person's health are concealed in body fluids such as blood and urine. So-called blood work and a urine test are sacrosanct to virtually every medical diagnosis.

One's blood chemistry is particular informing as to a person's state of health. The kidneys remove fluids, waste products and other substances from the blood. Thus, an analysis of the urine can reveal many important clues as to a person's health. Urinalysis is especially important in detecting urinary tract infections, chronic kidney disease and in monitoring diabetes.

Diabetes is a chronic health condition that affects how the human body turns food into energy.

As shown in FIG. 1, the stomach (Block 102) breaks down most of the food (Block 101) we eat into sugar, i.e., glucose. The stomach and small intestines (Block 103) absorb the glucose and then releases it into bloodstream 104.

Bloodstream 104 carries the glucose to the cells (Block 107) of the body for immediate conversion to energy if energy is currently needed. Block 107 is a representative cell of all of the cells in the body for purposes of this explanation.

If energy is not currently needed, the glucose is stored in the body (Block 106) until it is needed by the cells in order to produce energy.

The pancreas (Block 105) monitors the bloodstream and produces insulin in proportion to the amount of glucose present in the bloodstream. As shown in FIG. 1, insulin is the trigger which controls switch 108 inside the cell that allow glucose from the bloodstream to actually enter the cell for conversion to energy. Without insulin, the switch does not open and no glucose is allowed to enter the cell.

As mentioned above. Block 107 is a representative of all cells in the human body, thus each cell has a corresponding switch 108.

When the pancreas does not make a sufficient amount of insulin or the body cannot use all of the insulin the pancreas produces, the excess glucose, i.e., blood sugar, remains in bloodstream 104. This condition is known as diabetes and can lead to severe medication complications if not treated.

There are two primary types of diabetes, Type 1 and Type 2. People with Type 1 diabetes do not produce insulin and people with Type 2 diabetes do not respond to insulin as well as they should.

Diabetes treatment regiments often require constant monitoring of blood sugar. An analysis of the blood is often used for this purpose. Such an analysis usually requires a visit to a doctor's office or to a lab for blood to be drawn and analyzed. There are also over the counter consumer grade blood sugar monitors that can be purchased as well.

Analysis of the urine can also be used to monitor a diabetic condition. One such urine test measures the amount of a protein in the urine called microalbuminuria. This test detects when small amounts of albumin, the main protein in the blood, has found its way into the urine. This condition is indictive of kidney disease which can lead to kidney damage and eventually to kidney failure. Thus, the presence of albumin in the urine suggest that the kidneys are not working as well as they should and further investigation is warranted.

A second urine test for those with Type 1 diabetes measures the number of ketones in the urine. Ketones are produced by the body when the pancreas does not produce a sufficient amount of insulin to control body cells in order to allow glucose to enter cells in order to produce energy. The body compensates for the shortage of insulin by turning fat into energy for use by the cells. Ketones are toxic to the body and can cause severe medical complications.

While a number of products are known in the art that, to a least some degree, individually fulfil the need for toilet paper or alternative wipes and the need for urine testing for the purpose of self-administered medical screening, the art is devoid of any device or method that combines these necessary activities together in one product. The present invention meets this need.

### BRIEF DESCRIPTION OF THE DRAWINGS

The novel features of the present invention are set out with particularity in the appended claims, but the invention will be understood more fully and clearly from the following detailed description of the invention as set forth in the accompanying drawings in which:

FIG. 1 block diagram of how the humane body digests food into glucose and how glucose enters body cells;

FIG. 2 is a top view of one embodiment of a personal hygiene wipe with integral urine glucose detection feature in accordance with the present invention;

FIG. 3 is a cross-section taken alone line 3-3 in FIG. 2;

FIG. 4 is a diagram of one example of a chemical composition used in the present invention; and

FIG. 5 illustrates one embodiment of a container for the personal hygiene wipe with integral urine glucose detection feature in accordance with the present invention.

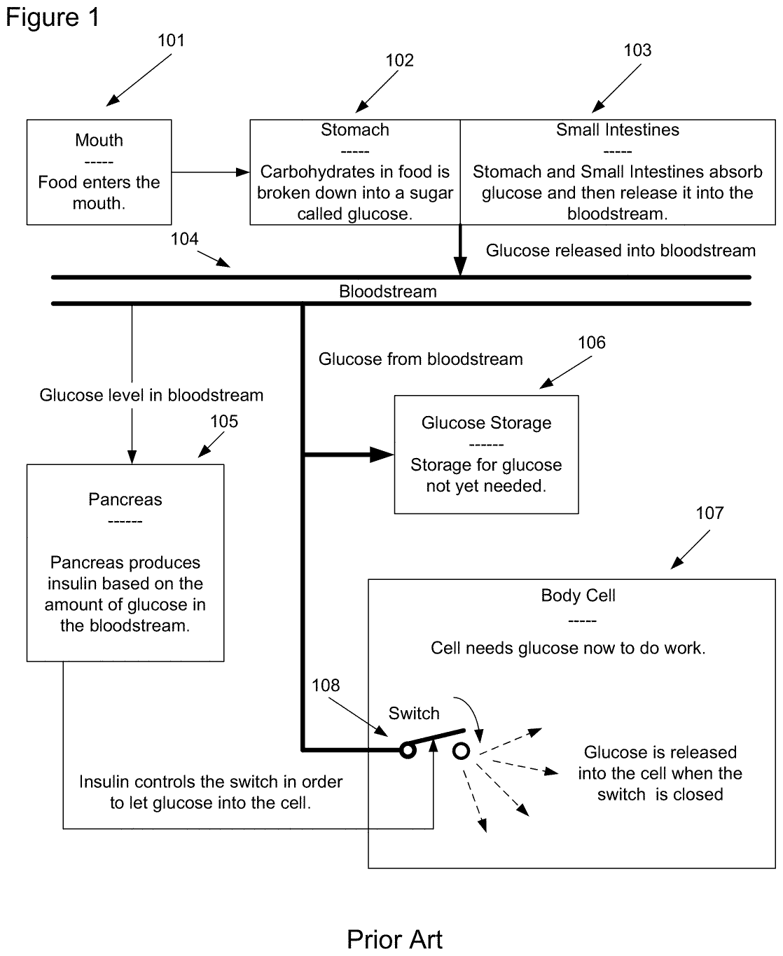

### DESCRIPTION OF THE PREFERRED EMBODIMENT

An example embodiment of the present invention will now be described with reference to the accompanying drawings.

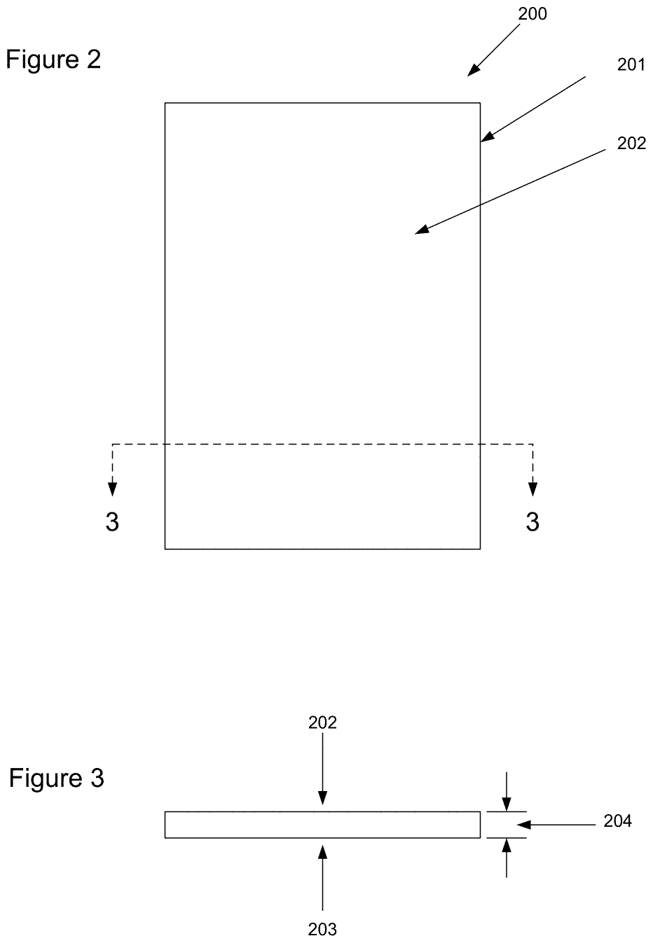

FIG. 2 is a top view of one embodiment of the personal hygiene wipe 200 of the present invention. The wipe includes a substrate 201. The substrate is the material which gives the wipe a physical structure and is used to hold urine glucose detection ingredients as will be discussed below. Substrate 201 has a top surface 202 and a bottom surface 203 as shown in FIG. 3.

FIG. 3 is a cross-section of substrate 201 taken along line 3-3 as shown in FIG. 2. Thickness 204 of substrate 201 is determined by the material used to form substrate 201 as well as its desired strength and rigidity. By way of example only, such thickness can range from 0.0254 to 0.254 millimeters.

Substrate 201 can be formed of a number of materials that are known in the art, including cotton, polyester, wood pulp and viscose. Cotton is soft to the touch and has liquid-absorbing and retaining properties. Polyester is made of plastic fiber and is known to have good strength. Wood pulp provides a wipe with a high surface area and a flat ribbon-like morphology which gives it high absorbency properties. Viscos is a type of rayon fabric made from wood pulp and has good absorbency, high strength and good softness. Other materials that are known in the art may be used as well.

As show in FIG. 2, substrate 201 is depicted as being generally rectangular in construction. However, the invention is not limited thereto. Substrate 201 may be formed in other shapes that are consistent with the objectives of the invention. Substrate 201 may be of a size that makes it convenient for packing a plurality of wipes in a user-friendly commercial wipes container for easy carrying and dispensing. Such a container is illustrated in FIG. 5.

By way of example only, subtract 201 may have size of 4 inches by 8 inches.

In order to complete construction of wipe 200, substrate 201 is saturated in a liquid comprising the active ingredients:
Glucose oxidase enzyme;
Catalase enzyme;
Iodide salt; and
Starch.
   Alternatively, the following ingredients can be used:
Glucose Oxidase enzyme; and
An Anthocyanin.

The proportions of each ingredient by mole weight are as follows:
Glucose Oxidase−1.875*10\hat{ }−10 mol/wt
Catalase−1.563*10\hat{ }−11 mol/wt
Potassium Iodide−6.024*10\hat{ }−4 mol/wt
Starch−˜7.401*10\hat{ }−7 mol/wt
Benzoic Acid−4.094*10\hat{ }−5 mol/wt** ** Made up to a pH of 5 with 6.4886*10\hat{ }−6 mol/wt of NaOH

The above ingredients may also be express by activity level as follows:
Glucose Oxidase−1.875*10\hat{ }−10 mol/wt activity−100 U/mg
Catalase−1.563*10\hat{ }−11 mol/wt activity−˜30,000 U/mL
Potassium Iodide−6.024*10\hat{ }−4 mol/wt
Starch−˜7.401*10\hat{ }−7 mol/wt
5 Benzoic Acid−4.094*10\hat{ }−5 mol/wt** ** Made up to a pH of 5 with 6.4886*10\hat{ }−6 mol/wt of NaOH

When the wipe of the invention comes into contact with urine, glucose in the urine which is above a predetermined level reacts with oxygen and water and is catalyzed by glucose oxidase to give gluconic acid and hydrogen peroxide. The hydrogen peroxide then oxidizes iodide ions to iodine, catalyzed by catalase. The iodine then forms a matrix with starch to produce a blue-black color to indicate to the user the present of glucose in the urine.

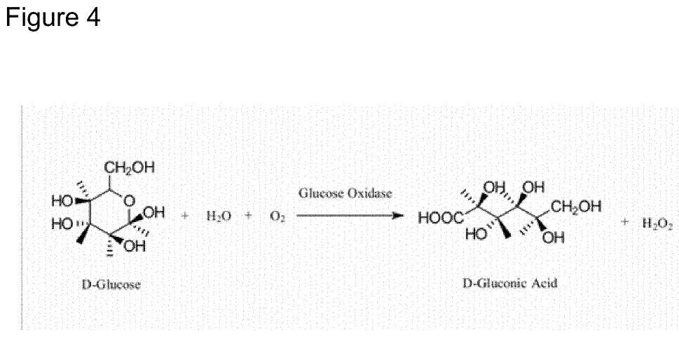

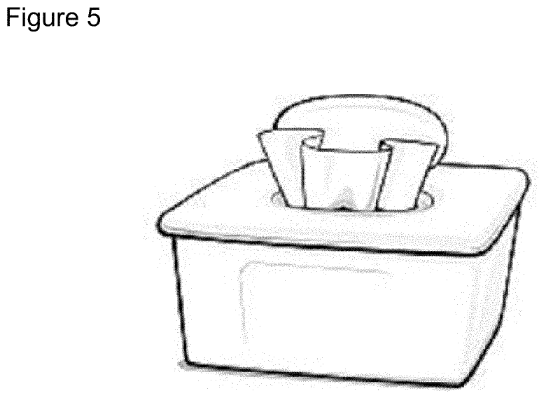

FIG. 4 is the structural formula of the above indigents.

With reference to FIG. 4, the first step detects glucose in the urine. This is the reaction of D-glucose with oxygen and water via glucose oxidase to produce D-gluconic acid and hydrogen peroxide. Glucose oxidase is an enzyme that is highly specific to glucose and will generally only catalyze this specific reaction:

(D-Glucose+2H₂O+O₂ Glucose Oxidase→D-Gluconic Acid+2H₂O₂)

The second step is where the indication of test results occurs. The hydrogen peroxide produced in step one is catalytically decomposed via the enzyme catalase into water and oxygen. The iodide ions likely donate electrons to this process, oxidizing to iodine solid. The iodine is then trapped inside the folds of the starch molecules producing the blue-black colour observed.

(2H₂O₂+2H⁺+2e⁻->2H₂O)

The presence of glucose in urine is called glycosuria or glucosuria. In a healthy person, glucose is not usually found in urine. However, glucose can still be present in urine and range between 0 to 0.8 mmol/l (0 to 15 mg/dL) and still be considered normal. The color change in the wipe of the present invention is designed to be triggered by a glucose level above 25 mg/dl.

Applicant has found that it takes only a few drops of urine for the inventive wipe to react and product a color change in the presence of glucose in the urine above the normal range.

As pointed out above, the substrate of the wipe of the present invention can be made of a material that is soft to the touch and has liquid-absorbing quantities. Thus, the wipe may be used by females to clean the vaginal area after urination and by males to clean the head of the penis as well. Accordingly, any glucose in the urine will cause the color of the wipe to change, thus indicating the presence of glucose.

The present invention provides a safe and convenient self-administered medical screening tool where the presence of glucose in urine is indicative of a possible medical condition. The services of a medical professional can then be sought for follow-up and further analysis of the condition.

The wipe of the invention may be used solely for the vaginal area or penis. In addition, it may also be used by males and females for cleaning the anus area after a bowel movement. When used in this way, the wipe of the invention provides both bathroom hygiene and self-administered medical screening using the same product.

While the foregoing specification teaches the principles of the present invention, with examples provided for the purpose of illustration, it will be appreciated by one skilled in the art from reading this disclosure that various changes in form and detail can be made without departing from the true scope of the invention.

I claim:

1. A hygiene wipe, said wipe comprising:
a substrate adapted for impregnation with a composition having one or more drying, cleaning, odor control, or antibacterial properties; and
a chemical composition for detecting glucose in urine, wherein said substrate is impregnated with said composition.

2. The hygiene wipe of claim 1, wherein said substrate is made of a material selected from the group consisting of cotton, polyester, wood pulp and viscose.

3. The hygiene wipe of claim 1, wherein when said wipe is subjected to glucose in urine, at least a portion of said wipe changes to a different color.

4. The hygiene wipe of claim 3, wherein said change in color persist after said wipe is no longer subjected to glucose.

5. The hygiene wipe of claim 1, wherein said composition is comprised of the ingredients of catalase enzyme, iodide salt, starch and benzoic acid.

6. The hygiene wipe of claim 5, wherein the proportion of said catalase enzyme in said composition by mole weight is 1.875*10\hat{ }−10 mol/wt.

7. The hygiene wipe of claim 6, wherein the proportion of said iodide salt in said composition by mole weight is 1.563*10\hat{ }−11 mol/wt.

8. The hygiene wipe of claim 7, wherein the proportion of said starch in said composition by mole weight is ˜7.401*10\hat{ }−7 mol/wt.

9. The hygiene wipe of claim 8, wherein the proportion of said benzoic acid in said composition by mole weight is 4.094*10\hat{ }−5 mol/wt.

10. The hygiene wipe of claim 1, wherein said composition is comprised of the ingredients of glucose oxidase enzyme and anthocyanin.
